(19) United States
(12) Patent Application Publication
(10) Pub. No.: US 2007/0026069 A1
(43) Pub. Date: Feb. 1, 2007

(54) Biommetic hierarchies using functionalized nanoparticles as building blocks

(76) Inventors: Venkatram P Shastri, Nashville, TN (US); I-Wei Chen, Swarthmore, PA (US); William Zindarsic, Philadelphia, PA (US)

Correspondence Address: RATNERPRESTIA, P O BOX 980, VALLEY FORGE, PA, 19482-0980, US

(21) Appl. No.: 10/550,923

(22) PCT Filed: Mar. 26, 2004
(86) PCT No.: PCT/US04/09192

§ 371 Date: Sep. 22, 2006

Related U.S. Application Data
(60) Provisional application No. 60/458,258, filed on Mar. 28, 2003.

(51) Int. Cl. A61K 35/12; A61K 9/14

(52) U.S. Cl. 424/486; 424/489; 424/93.7; 977/906

(57) ABSTRACT

The invention provides a three-dimensional construct including a polymeric matrix and a nanoparticle as shown in FIG. 1 having a diameter of about 5 nm to about 10 microns, wherein the nanoparticle is (a) coated with at least two monomolecular layers each carrying biological information and (b) dispersed in the polymeric matrix at a density of at least 0.01 vol %. The invention further provides a method of presenting biological information to a cell or a tissue and thereby affecting at least one parameter of the cell or the tissue, the method involves providing the three-dimensional construct and contacting it with the cell or the tissue to present the biological information and thereby affecting at least one characteristic of the cell or the tissue. In certain embodiments, the diameter, the biological information and the density are selected to affect at least one characteristic of the cell or the tissue.

### BACKGROUND OF THE INVENTION

1. FIELD OF INVENTION

This invention relates to building three dimensional constructs or biomimetic hierarchies using nanoparticles carrying biological information. This invention also relates to a method of presenting biological information to a cell or a tissue.

2. DESCRIPTION OF RELATED ART

Biological polymers such as collagen and hyaluronic acid have been utilized to fabricate scaffolds for regeneration of dermal tissue and skeletal components such as bone and cartilage. A non-polymeric bioactive material such as hydroxyapatite has been utilized in various implant applications due to its similarity with mineral constituents found in hard tissues (e.g., teeth and bones) and cartilage. One way to prepare hydroxyapatite from an aqueous solution has been reported by Riman et al in Solution Synthesis of Hydroxyapatite Designer Particulates, Solid State Ionics, 151, (2002), 393-402. Hydroxyapatite has been used in combination with various substances such as, for example, collagen and silica. Li et al. demonstrated an apatite forination from a simulated body fluid (i.e., human blood plasma) on a pure silica gel (see Apatite Formation Induced by Silica Gel in a Simulated Body Fluid, J. Am. Ceram. Soc., 75, 2094-97, (1992)). A collagen-hydroxyapatite composite, COLLAGRAFT, in association with marrow elements is extensively used to repair fractures. Injectable, radiation curable polymers derived from poly(anhydrides) and poly(ethylene glycol)s (PEG) have been explored in tissue regeneration and reconstruction. Woven and non-woven meshes and cellular solids of biodegradable polymers are used in neo-tissue engineering. Other examples include a collagen complex with glycosaminoglycans used in dermal regeneration. These composites lack control over microstructure at the nanoscopic level.

Further, coating of a surface of an implant or a scaffold is one way to condition this surface to accommodate cell attachment and development. Moreover, such surfaces can have bioactive molecules localized on the surface. Conventional coating techniques are poorly defined at the sub-micron level, however, and do not provide a suitable bio-mimetic interface for attaching cells. Furthermore, known coatings typically yield a surface lacking chemical reactivity that is needed for the immobilization and presentation of bioactive molecules. Moreover, known coatings do not have versatility and control over surfaces at the nano-ranges.

Coating of surfaces using silicon dioxide is described by Stober et al., “Controlled Growth of Monodisperse Silica Spheres in the Micron Size Range,” J. Colloid Interface Sci., 26, 62-69(1968). This reference does not disclose coating of surfaces using modified or functionalized colloidal silica. Other related technologies and background are described in the following publications: E. P. Plueddemann, “Silane Coupling Agents,” Plenum Press, New York, Chapter 3, 49-73 (1982) and Vrancken et al., “Surface Modification of Silica Gel with Aminoorganosilanes,” Colloids and Surfaces, 98 235-241 (1995).

Polymeric colloidal particles are typically prepared by one of the three methods. In the method of emulsification-solvent evaporation, the polymer is dissolved in chlorinated hydrocarbon (organic solvent) such as methylene chloride or chloroform as disclosed by Wise, Donald L. ed., Handbook of Pharmaceutical Controlled Release Technology, Marcel Dekker Incorporated, New York, N.Y., pages 329-344 (2000). The polymer solution is then mechanically dispersed in an aqueous solution containing a polymeric surfactant, such as polyvinyl alcohol (PVA) or carboxymethoxycellulose (CMC), by homogenization or ultrasonication to form a microemulsion. The thermodynamically unstable microemulsion is stabilized by the presence of PVA. The organic solvent is then evaporated and the colloids (and/or NPs) collected by centrifugation to remove the excess PVA and then resuspended in a solution of interest.

Niwa et al. have developed a method to produce polymeric colloidal particles by first dissolving the polymer in a mixture of chlorinated hydrocarbon such as methylene chloride and acetone, and then pouring this solution into a aqueous phase containing PVA with mechanical stirring. (See Controlled Rel., (25), 89-98 (1993)). Acetone is added to enhance the difflusion of the methylene chloride solvent into the water phase. Like the solvent evaporation approach the organic solvent is evaporated and the colloids are separated from the PVA phase by centrifugation. Their approach is called spontaneous emulsification solvent diffusion (SESD).

Murakami et al. have reported a modification of the SESD procedure that relies on the gelation of the PVA phase around the emulsion droplets for stabilization ofthe colloids as they form in solution. (See Intl. J. Pharm., (187), 143-152 (1999)). In this approach, to control and restrict the gelation of PVA to the surface of the emulsion droplet, alcohol (ethanol or methanol), which is a solvent for PVA but a non-solvent for the polymer was used. The mechanism of colloid formation is again dependent on the presence ofthe polymeric emulsifier, PVA. This method yields colloids of mean diameter of above 260 nm.

Coatings of flat surfaces with multi-layers including synthetic and natural polymers have been studied for many years. Also, attempts to provide multiple layers onto colloids have been reported. Sukhorukov et al. describe using colloids as templates for a polyelectrolyte multi-layered formation (see Stepwise Polyelectrolyte Assembly on Particle Surfaces: A Novel Approach to Colloid Design, Polymers for Adv. Technologies, 9, 759-767(1996)). G. Decher describes electrostatically driven assembly of multi-layered structures on colloids (see Fuzzy Nanoassemblies: Toward Layered Polymeric Multicomposites”, Science, 277, 1232-1237 (1997)). These articles do not describe dispersing multi-layered formations in a scaffold or another three dimensional media.

Attempts have been made to absorb biomolecules onto nanoparticles. However, biological activity of many biological molecules is directly linked to their conformation and adsorption can cause changes in conformation. (See J. N. Lindon,E. W. Salzman, Does the Conformation of Adsorbed Fibrinogen Dictate Platelet Interactions With Artificial Surfaces?, Blood, Vol 68, No2, 355-362, (1986)).

Bio-ceramics that mimic bone structure and are derived from collagen and hydroxyapatite (e.g., COLLAGRAFT) have been used in association with marrow elements to successfully treat fractures. COLLAGRAFT is not suitable for usage in situations that require retention of three-dimensional structure such as facial reconstructions and load bearing situations such as fractures of the long bones. Biodegradable, injectable and curable polymers derived from poly(anhydrides), PEG and poly(α-hydroxyacids), while capable of retaining their geometry over extended periods of time, lack any biological functionality or well-defined nanoscaled architecture. In the context of bone regeneration, ceramic scaffolds derived from calcium phosphate such as INTEPORE ceramic lack any biological information. Furthermore, neither COLLAGRAFT nor any of the above mentioned polymers or ceramic scaffolds offer control over the microstructure at the nanoscale. Indeed, their properties are rather inhomogeneous and depend on processing conditions and process related variabilities. Cellular responses can be sensitive to this lack of homogeneity at the nanoscopic levels because the size-scale of receptors clusters and domains on cell surfaces is often in the same nanoscale size range as the size of scaffolds. Therefore, there is a need in the art for new compositions and methods to provide three-dimensional constructs having bio-functionalities with nanoscopic control.

All references cited herein are incorporated herein by reference in their entireties.

### BRIEF SUMMARY OF THE INVENTION

Accordingly, the invention provides a three-dimensional construct comprising a polymeric matrix and a nanoparticle comprising a structure and a chemical functional group attached to the structure, wherein the nanoparticle has a diameter of about 5 nm to about 10 microns and is (a) coated with a monomolecular layer carrying biological information and (b) dispersed in the polymeric matrix at a density of at least 0.01 vol %.

In certain embodiments ofthe invention, nanometer-sized colloids possessing a desired surface chemistry and charge are used as the template starting material. Using silica colloids as a non-limiting example, the inventors have demonstrated the feasibility of the invention. The silica colloids obtained by this technology have a typical diameter of about 10-5000 nm and preferably a monodispersed narrow size distribution. The amine group on the colloidal- particle surface can be coupled to other functional groups, synthetic or natural polymers, and biomolecules such as, for example, genes, proteins, growth factors and other bio-functional moieties by, for example, covalent bonding, lidand-substrate binding and electrostatic adsorption. Binding of various molecules to the nanoparticle can be repeated to build up multiple layers of functionality of very precise thickness desired in various applications (e.g., tissue engineering). Upon achieving an appropriate functionalization or coating, other bioactive layers such as, for example, hydroxyapatite may be deposited to enhance response to bone cells. Once the desired biomimetic nano-structure is evolved, these biomimetic nanoparticles can be dispersed in a polymeric matrix and then formed into gels, fibers, meshes and solids to form the three dimensional construct ofthe invention. It can be formed into shapes by standard polymer forming processes, such as extrusion, molding, pouring, electrospinning, spin coating, stamping, 3 dimensional printing and other methods known in the art. Alternatively, such biomimetic nanoparticles can be used to coat surfaces of biocompatible constructs to impart or enhance their biofunctionality.

Also, the invention provides a method of presenting biological information to a cell or a tissue, the method comprising providing the three-dimensional construct of the invention and contacting the three-dimensional construct with the cell or the tissue to present the biological information and thereby affecting the at least one characteristic of the cell or the tissue. In certain embodiments, the diameter, the biological information and the density are selected to affect the at least one characteristic of the cell or the tissue.

Further, the invention provides a method of making the three-dimensional construct of the invention, the method comprising providing the polymeric matrix, providing an unprocessed nanoparticle, making the nanoparticle by contacting the unprocessed nanoparticle with a carrier of biological information to form the innermost monomolecular layer and dispersing the nanoparticle in the polymeric matrix at the density of at least 0.1 vol. %.

The term “unprocessed particle” as used herein means a particle having requisite chemical functional groups but not yet covered with monomolecular layers of biological information. Carriers of biological information as used in this disclosure are substances with can impart requisite biomolecules, polymers and bone substitutes. Non-limiting examples of such carriers are collagen, poly(acrylic acid) and a mixture of nitrate tetrahydrate and ammonium phosphate.

Also, the invention provides a nanoparticle comprising a structure, said structure is being a member selected from the group consisting of silicon oxide functionalized with a chemical functional group, poly(lactic acid), poly(lactic-co-glycolic acid), and poly(anhydride), a monomolecular layer of hydroxyapatite, and optionally a monomolecular layer of poly(acrylic acid) and/or a monomolecular layer of collagen, wherein the structure is coated with the monomolecular layer of hydroxyapatite and optionally with the monomolecular layer of poly(acrylic acid) and/or the monomolecular layer of collagen, provided that the monomolecular layer of hydroxyapatite is an outermost monomolecular layer.

Additionally, the invention provides method of administering nanoparticles to a cell, the method comprising providing nanoparticles, optionally providing an auxiliary surface, wherein the auxiliary surface is a polymer, a carbonaceous material, a wool, a glass, a ceramic, or a metal and wherein the auxiliary surface is in communication with the nanoparticle and contacting the cell with the nanoparticle.

### BRIEF DESCRIPTION OF SEVERAL VIEWS OF THE DRAWINGS

The invention will be described in conjunction with the following drawings in which like reference numerals designate like elements and wherein:

FIG. 1 is a flow chart for the biomimetic assembly on nanoparticles.

FIG. 2 is Scanning Electron Microscopy (SEM) micrographs of (A) the silica nanoparticles (SiO₂) and (B) amine functionalized silica nanoparticles following poly(acrylic acid) adsorption (SiO₂ —NH₂ —PAA).

FIG. 3 is a graph showing zeta potential measurements as a function of pH for silica nanoparticles (SNPs) and functionalized silica nanoprticles (FSNPs) including nanoparticles functionalized with aminopropyltriethoxysilane (APS FSNPs) and nanoparticles further coated with poly(acrylic acid) (PAA FSNPs) FIG. 4 is a SEM micrograph of amine functionalized silica nanoparticles following poly(acrylic acid) (PAA) adsorption and collagen adsorption (SiO₂ —NH₂ —PAA-Collagen).

FIG. 5 is a graph showing zeta potential measurements as a function of pH for collagen, APS FSNP, PAA FSNP, APS FSNP following collagen adsorption (APS/collagen FSNP) and PAA FSNP following collagen adsorption (PAA/collagen FSNP).

FIG. 6 is a graph showing % yield of hydroxyapatite (HAp) as a function of reactant concentration and pH.

FIG. 7 is a SEM micrograph of the amine functionalized silica nanoparticles coated with poly(acryl amine) (PAA FSNP) sequentially coated with collagen and HAp

FIG. 8 is the X-ray diffraction pattern of PAA FSNP following collagen adsorption and HAp coating.

FIG. 9 is a spectrum of the energy versus relative counts obtained in the Energy Dispersive Spectroscopy analysis of PAA FSNP following collagen adsorption and HAp coating.

FIG. 10 is a graph showing zeta potential measurements as a function of pH for HAp and PAA FSNP with adsorbed collagen before and after HAp coating.

FIG. 11 shows optical micrographs of alginate gel containing PAA FSNP following collagen adsorption and HAp coating at different magnification, wherein FIG. 11A shows the micrographs on a scale of 50 micron, and FIG. 11B shows the micrographs on a scale of 10 micron.

### DETAILED DESCRIPTION OF THE INVENTION

The invention flows from the discovery that a three-dimensional construct comprising a nanoparticle dispersed in a polymeric matrix, wherein the nanoparticle is coated with a monomolecular layer comprising biological information can be used to present this information to a cell or a tissue in a predictable and controllable manner. Inventors have discovered that nanoparticles can be constructed to have a desirable size and characteristics and further applied in combination with various materials and surfaces to a call or a tissue to cause a desired effect.

Accordingly, the invention provides a three-dimensional construct comprising a polymeric matrix and a nanoparticle comprising a structure and a chemical functional group attached to the structure, wherein the nanoparticle has a diameter of about 5 nm to about 10 microns and is (a) coated with a monomolecular layer carrying biological information and (b) dispersed in the polymeric matrix at a density of at least 0.01 vol %. The three dimensional construct of the invention can be used to fabricate bone graft substitutes, interactive scaffolds for tissue engineering and organ regeneration, cell-culture substrates, implant coatings, and sutures. It can also be administered (e.g., injected, swallowed, or inhaled) in a liquid state or as a coating on an auxiliary surface. The uniqueness of this invention is that it controls the microstructure and functionality at the nanoscopic levels, by using colloidal particles in the early steps of the functionalization and self-assembly. In the invention, biological information such as chemical and biological functionalities is imparted by anchoring them onto nanoparticles. These functionalities include a primary or a secondary layer of organic or inorganic coatings onto nanoparticles, or biologically relevant moieties such as peptide, proteins, genetic material or other chemical entities that are tethered to the nanoparticles to alter and/or modify the biological and cellular response to the surface of the biomimetic hierarchies. Further, such nanoparticles are dispersed in, for example, a curable polymeric matrix to provide the three dimensional construct of the invention. The invention has a wide-ranging applicability in areas of tissue engineering, medical devices, medical implants, bio-MEMS (micro electro-mechanical systems), and high throughout screening technologies.

As a non-limiting example, nanoparticles of inorganic oxides such as silicon oxide were prepared by a sol-gel process and functionalized to bear amine groups covalently bound to the surface of nanoparticles and thereby imparting a positive charge. Next, a molecular layer of biocompatible synthetic polymer bearing an opposite charge (negative charge), e.g., poly(acrylic acid) was assembled onto the colloidal particles in solution under appropriate pH to render the surface negatively charged. This negatively charged colloidal surface was further modified by a monomolecular layer of a biologically relevant natural polymer, e.g., collagen via electrostatic assembly to introduce bio-recognition. The layer of collagen can be then used to bind biomolecules such as, for example, growth factors, peptides, and nucleotides by a non-covalent approach. In addition, such biological layer offers cellular binding sites (e.g., RGD) and a natural surface for deposition of bio-ceramics such as hydroxyapatite.

The resultant biomimetic-colloids can then be either self assembled or co-assembled in various environments including gels (e.g., hyaluronic acid, alginate); polymers (thermoplastics and radiation curable), ceramics (e.g., tri-calcium phosphate) using well-established techniques such as dispersion; co-extrusion; solvent-casting; stereo-, UV- and e-beam-lithography; free form powder fabrication; and others known in the art into objects of desirable geometry and functions. The assembly can also be further modified by incorporating cells, grow factors, genes, therapeutic agents, mechanical reinforcement, and other moieties of functional purposes. Since the process starts with a plurality of nanoparticles with built-in functionality embodied by a covalently bonded coating, biomolecules and other functional moieties are reproducibly presented on each nanoparticles, thus offering an extraordinarily precise control over the biomimetic assembly process at the nanoscale. In addition to offering control at the nano-scale, the process is extremely robust and scalable because it is based on colloidal processing. The functionalized colloids are not limited to oxides but can include synthetic degradable and non-degradable polymer. These polymers can be pre-functionalized before processing into colloids, functionalized during the processing step by physical blending with other functionalized polymers, or functionalized post-processing. Processing in this context is the formation of the template colloidal particles. The invention will be further described in detail below.

Nanoparticle

The nanoparticle of the invention has an inorganic or organic structure (or a platform) which is functionalized with a chemical group or a plurality of chemical groups and is coated with at least one monomolecular layer. In certain embodiments, the inorganic structure is an oxide, a nitride, a carbide, calcium silicate, calcium phosphate, calcium carbonate, a carbonaceous material, a metal, or a semiconductor. Non-limiting examples of oxides are Al₂O₃, TiO₂, ZrO₂, Y₂O₃, SiO₂, ferric oxide, ferrous oxide, a rare earth metal oxide, a transitional metal oxide, mixtures thereof and alloys thereof Non-limiting examples of metals are aluminum, gold, silver, stainless steel, iron, titanium, cobalt, nickel, and alloys thereof. In certain embodiments, the organic structure is selected from the group consisting of biodegradable polymers, non-biodegradable water-soluble polymers, non-biodegradable non-water soluble polymers, lipophilic moieties, and biopolymers. Non-limiting examples of the organic structure are poly(styrene), poly(urethane), poly(lactic acid), poly(glycolic acid), poly(ester), poly(alpha-hydroxy acid), poly(ε-caprolactone), poly(dioxanone), poly(orthoester), poly(ether-ester), poly(lactone), poly(carbonate), poly(phosphazene), poly(phosphonate), poly(ether), poly(anhydride), mixtures thereof and copolymers thereof.

The chemical functional group is preferably attached to the nanoparticle's surface by forming a covalent bond. In certain embodiments, the chemical functional group is a member selected from the group consisting of an amine group, a hydroxyl group, a carboxy group, an —OSO₃H group, a —SO₃H group, a —SH group, an —OCN group, a phosphorous group, an epoxy group, a vinylic moiety, a silane coupling agent, an acrylate, a methylacrylate, a metal alkoxy group, and derivatives thereof. Preferably, the chemical functional group is an amine group. Chemical functional groups can be attached to the nanoparticle by, for example, methods Icnown in the art. When the nanoparticle has the organic structure, the chemical groups can come from monomeric units prior to polymerization or the polymer's existing chemical groups can be modified to have different desired chemical groups.

The nanoparticles of the invention are preferably are in a colloidal preparation and made by any method suitable for obtaining a colloid, as described, for instance, by Morrison and Ross, COLLOIDAL DISPERSIONS: SUSPENSIONS, EMULSIONS AND FOAMS (Wiley Publ. 2002). Then, the preparation can be chemically treated to impart a functional group to the nanoparticle's surface such as, for example an amine group as described in detail by Chen et al. in a co-pending U.S. patent application Ser. No. 10/427,242 filed on May 1, 2003, titled “A Nanometer-Sized Carrier Medium” and also in a co-pending U.S. patent application Ser. No.10/668.484 filed by Shastri et al. on Sep. 22, 2003.

Accordingly, nanometer-sized colloidal oxides can be prepared through base-catalyzed hydrolysis and condensation. The silica colloids obtained by this technology have a diameter of about 10 to about 5000 nm. These colloids possess a monodispersed narrow size distribution. The colloid formation is controlled at pH about 3 to about 5 before the surface modification because silanol groups of aminosilane in aqueous solution are relatively stable in acidic conditions. Since the silanol groups have an isoelectric point about 2 to about 3, the aminosilane in the pH range of about 3 to about 6 exits as zwitterions. The formation of zwitterions prevents the continuous hydrolysis and condensation reaction of aminosilane. A water-stable amine-terminated oxide is prepared by blocking consecutive reactions of aminosilane in the aqueous condition. The nanometer-sized, uniform, amine-terminated oxide suspension is washed and stored as the source material for later use.

In certain embodiments of the invention, the nanoparticle is silica colloid functionalized with an aminosilane such as, for example, tetraethylorthosilicate (TEOS) as described by Chen et al. and Shastri et al. The size of silica can be controlled by the initial reagent concentration, reaction time and solvent. The amine-functionalized colloidal silica provides a platform to build additional layers of biological information

In the invention, the nanoparticles are further treated to provide at least one monomolecular layers of biological information. If a plurality of layers is provided, they would be deposited sequentially so that an innermost monomolecular layer is attached to or in contact with the nanoparticle. If there are only two layers, the outermost monomolecular layer is attached to or in contact with the innermost layer. If more than two layers are provided, the layer(s) disposed between the outermost layer and the innermost layer, thereafter referred to as an intermediate monomolecular layer, is (are) attached to or in contact with at least one of the innermost monomolecular layer and the outermost monomolecular layer. The complete coverage of the nanoparticle'surface as well as the complete coverage of each of the sequential layers is preferred, however, the incomplete coverage is also acceptable. Formation of layers can be monitored by measuring zeta potential as described by Sukhorukov et al, supra. Zeta potential is a voltage difference between the surface of the particle and the solvent beyond the outer layer. The goal in most formulations is to maximize zeta potential which would prevent particle-particle agglomeration and keep the dispersion uniform. Zeta potential also depends on pH as shown in FIGS. 3, 5, and 10.

In certain embodiments, the contact between layers and the nanoparticle as well as between the innermost layer and the nanoparticle was made possible by at least one of the mechanisms including covalent bonding, hydrogen bonding, ionic or electrostatic bonding, Van der Waals forces and ligand-substrate binding. Types of mechanisms can also change from, for example, electrostatic bonding to hydrogen bonding depending on the pH. A non-limiting example of covalent bonding is a reaction between amine groups of a nanoparticle (e.g., amine functionalized silicon oxide nanoparticle) with poly(acrylic acid) to form amide bonds.

Hydrogen bonding is a strong electrostatic attraction between two independent polar molecules, i.e., molecules in which the charges are unevenly distributed, usually containing nitrogen, oxygen, or fluorine. These elements have strong electron-attracting power, and the hydrogen atom serves as a bridge between them. The hydrogen bond is much weaker than the ionic or covalent bonds. A non-limiting example of hydrogen bonding is bonding between carboxylic groups at lower pH.

A non-limiting example of ionic bonding is a polyanion/polycation assembly such as, for example, collagen/hydroxyapatite, collagen/poly(acrylic acid), poly(allylamine hydrochloride)/poly(styrene sulfonate), poly(diallyldimethyl ammonium chloride)/ poly(styrene sulfonate), and poly(diallyldimethyl ammonium chloride)/DNA (see Sukhorukov et al., supra).

Long-range forces, or so-called Van der Waals forces, account for a wide range of physical phenomena, such as friction, surface tension, adhesion and cohesion of liquids and solids, and viscosity. Van der Waals forces arise in a number of ways such as, for example, the tendency of electrically polarized molecules to become aligned. A non-limiting example of Van der Waals forces useful in this invention is a nanoparticle bearing an alkyl functionality and having lipophilic molecules adsorbed onto its surface. The alkyl functionality can be introduced by reaction of an amine group with an aliphatic carboxylic acid such as decanoic acid so that this hydrophobic layer can be used to adsorb other hydrophobic species via Van der Walls interactions.

A non-limiting example of ligand-substrate binding is binding between an antibody and antigen pair and biotin (e.g., a biotinylated antibody) and avidin (e.g., streptavidine) interactions.

One of the purposes of having the monomolecular layers on the nanoparticle is to carry biological information to a cell or a tissue at the contact of such nanoparticle with the cell or the tissue. The term “biological information” as used herein means the information carried by a monomolecular layer of the nanoparticle to a cell or a tissue which is capable of affecting at least one characteristic of the cell or the tissue such as, for example, proliferation and differentiation. Non-limiting examples ofbiological information are a biomolecule, a polymer, and a bone substitute. Non-limiting examples of the biomolecule is a bioactive polypeptide, a polynucleotide coding for the bioactive polypeptide, a cell regulatory small molecule, a peptide, a protein, an oligonucleotide, a nucleic acid, a poly(saccharide), an adenoviral vector, a gene transfection vector, a drug, and a drug delivering agent. In certain embodiments, the bioactive polypeptide is a growth factor, and such growth factor is a member selected from the group consisting of an epidermal growth factor, an acidic fibroblast growth factor, a basic fibroblast growth factor, a glial growth factor, a vascular endothelial growth factor, a nerve growth factor, a chondrogenic growth factor, a platelet-derived growth factor, a transforming growth factor beta, an insulin-like growth factor, a hepatocyte growth factor, a brain derived growth factor, bone morphogenic proteins and osteogenic proteins. In certain embodiments, the polymer is a member selected from the group consisting of poly(carboxylic acid), poly(sulphonic acid),poly(lysine), and poly(allylamine). Preferably, the poly(carboxylic acid) is poly(acrylic acid). In certain embodiments, the bone substitute is a member selected from the group consisting of a calcium phosphate, a bioactive glass composition and a bioceramic.

Non-limiting examples of calcium phosphates are hydroxyapatite, tricalcium phosphate, tetracalcium phosphate, and octacalcium phosphate. More examples can be found in Riman et al., supra and U.S. Pat. No. 6,331,312 to Lee et al. Non-limiting examples of bioactive glass composition are compositions including SiO₂, Na₂O, CaO, P₂O₅, Al₁₂O₃ and/or CaF₂, which can also be used in combination with calcium phosphates and/or bioceramics. Non-limiting examples of bioceramic are beta tricalcium phosphate, calcite, and diphosphonate.

Each monomolecular layer carries different biological information, however, it is also possible that more than one layer would carry similar or identical information.

The monomolecular layer can be provided by, for example, methods known in the art and may vary depending on desired configuration. Non-limiting examples of such methods are consecutive adsorption of polyanions and polycations as described by Decher, supra and modification of silica particles with poly(acrylic acid) as described by Suzuki et al., supra.

In certain embodiments, the nanoparticle can be applied onto an auxiliary surface as described further below.

Three Dimentional Construct

The three-dimensional construct of the invention comprises the nanoparticles as described above dispersed in a polymeric matrix at a density of at least 0.01 vol %. In certain embodiments, the nanoparticles are dispersed at the density of about 0.1 to about 5 vol %; in other embodiments, the density is about 1 to about 50 vol %.

Dispersion of nanoparticles in the polymeric matrix can be heterogeneous or homogeneous. In some cases, for example, when using a 3-D printing, it may be preferred to localize the particles of a certain type to generate a three dimensional construct with spatially well-defined information bearing nanoparticles. In certain embodiments, homogeneous dispersion is preferred, for example in applications requiring an equal distribution of bioinformation.

In certain embodiments, the polymeric matrix is a member selected from the group consisting of alginate, hyaluronic acid, poly(ethylene glycol), poly(vinyl alcohol), collagen, a peptide, poly(ethylene oxide)-b-poly(propylene oxide)-b-poly(ethylene oxide), poly(acrylic acid), and poly(isopropyl amide). In certain embodiments, the three-dimensional construct of the invention further comprises a cell dispersed within the polymeric matrix, wherein the cell can be at least one of chondroblast, chondrocyte, fibroblast, an endothelial cell, osteoblast, osteocyte, an epithelial cell, an epidermal cell, a mesenchymal cell, a hemopoietic cell, an embryoid body, a stem cell, and dorsal root ganglia.

In certain embodiments, the three-dimensional construct is in a form of a gel, a cross-linked polymer, a liquid, a foam, a sponge, a mesh, a solid particulate, a fiber or a layer. The form of the construct can be achieved by methods known in the art, for example by adding a chemical substance such as a gelation agent (e.g., a calcium salt) or a curing agent, or by solidifying by physical methods such as radiation. The three-dimensional construct can be further molded into a desired shape by extruding, electrospinning, 3-D printing and other methods known in the art.

In certain embodiments, the three-dimensional construct of the invention is prepared utilizing nanoparticles comprising silicon oxide functionalized with amine groups coated with a monomolecular layer of hydroxyapatite In certain embodiments, the three-dimensional construct of the invention is prepared by utilizing nanoparticles comprising silicon oxide functionalized with amine groups and coated with a monomolecular layer of collagen (the innermost monomolecular layer) and a monomolecular layer of hydroxyapatite (the outermost monomolecular layer). In certain embodiments, the three-dimensional construct of the invention is prepared utilizing nanoparticles comprising silicon oxide functionalized with amine groups and coated with a monomolecular layer of poly(acrylic acid) (the innermost monomolecular layer), a monomolecular layer of collagen (the intermediate monomolecular layer) and a monomolecular layer of hydroxyapatite (the outermost monomolecular layer), wherein the innermost monomolecular layer is attached to or in contact with the intermediate monomolecular layer and the outermost monomolecular layer is attached to or in contact with the intermediate monomolecular layer. In these embodiments, the three dimensional construct preferably comprises the above nanoparticle dispersed in an alginate gel, crosslinked poly(ethylene glycol), crosslinked poly(vinyl alcohol), collagen gel, a peptide gel, or poly(ethylene oxide)-b-poly(propylene oxide)-b-poly(ethylene oxide) polymer derived gel, and most preferably the polymeric matrix is an alginate gel. Gelation, cross-linking, curing or another type of hardening of the polymeric matrix is performed after dispersing the layered nanoparticle in the polymeric matrix and is made by methods known in the art.

In certain embodiments, the three-dimensional construct of the invention is prepared utilizing nanoparticles comprising at least one of poly(lactic acid), poly(lactic-co-glycolic acid), and poly(anhydride) coated with a monomolecular layer of poly(acrylic acid) and/or the monomolecular layer of collagen as the innermost monomolecular layer and the monomolecular layer of hydroxyapatite as the outermost monomolecular layer. In these embodiments, the polymeric matrix can be PMMA or any other suitable matrix as described above.

In any of the embodiments, more than one monomolecular layer of each kind can be provided if desired.

Moreover, the three-dimensional construct of the invention can further include a cell dispersed within the polymeric matrix. Non-limiting examples of such cells are chondroblast, chondrocyte, fibroblast, an endothelial cell, osteoblast, osteocyte, an epithelial cell, an epidermal cell, a mesenchymal cell, a hemopoietic cell, an embryoid body, a stem cell, and dorsal root ganglia.

Preferably, hydroxyapatite is deposited onto collagen from an aqueous mixture comprising calcium nitrate tetrahydrate and ammonium phosphate at a molar ratio about 1.5 to about 2.0 and pH of about 7 to about 9.5. In certain embodiments, pH is from about 7 to about 8 and in other embodiments, pH is from about 8 to about 9. Preferably, the molar ratio of calcium nitrate tetrahydrate to ammonium phosphate equals 2.

The three-dimensional construct of the invention can be prepared by mixing or otherwise dispersing the nanoparticles coated with monomolecular layers of biological information in the desired polymeric matrix (e.g., alginate, PMMA, etc) at the density of at least 0.1 vol %. Next, depending on the choice of the polymeric matrix, the dispersion can be further treated to fixate the nanoparticles in the polymeric matrix, for example, by adding an agent (e.g., calcium chloride or ammonium hydroxide) to induce gelation, by cross-linking or curing. Fixation of the matrix can also be done utilizing non-chemical methods such as, for example, heating or irradiation.

Preparation of the nanoparticle and the three-dimensional construct of the invention is described herein with silica oxide as the structure.

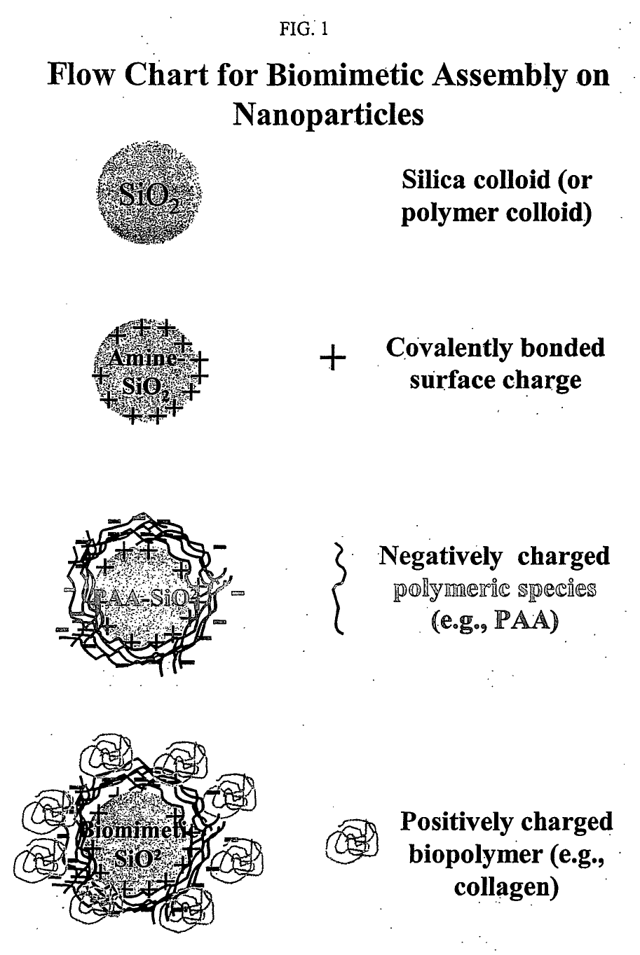

A flow chart of the method of preparing multifunctional colloidal silica with poly(acrylic acid), collagen and hydroxyapatite coating is illustrated in FIG. 1. The assembly ofpoly(acrylic acid) (PAA) and collagen is achieved by principles of electrostatic binding. In this method, the starting colloidal templates are silica particles with surface amine groups that render the surface of the colloids positively charged at an appropriate pH. Similarly, at a pH typically around 4-5, the carboxylic acid groups in the PAA are ionized giving the PAA molecule an excessive net negative charge. PAA can thus be electrostatically attracted to the silica-amine surface leading to a surface deposition and assembly of PAA on the amine-rich colloidal silica surface. This in turn causes the reversal of the surface charge to negative, which can then be used to assemble a secondary molecular layer of positively charged moieties such as collagen. This assembly is carried out at a pH of 4-5 when the net charge on the collagen molecule is positive. The above distinct steps resulting in precise molecular level deposition and modification can be repeated with other appropriate molecules to achieve surfaces tailored at the molecular level. The progress of the process can be monitored using photon-correlation-spectroscopy for particle size determination, and zeta meter for surface potential determination. In addition, direct verification of a proteinaceous coating such as collagen can be provided by micro-BCA protein assay. The typical shape of the starting and final particles is spherical with very similar diameters that can be varied in the range from tens to hundreds of nanometers. This is because the size of poly(acrylic acid) and collagen is quite small and does not significantly add to the size of the colloid unless the latter is less than 10 nm. As a result, a very precise size control of the final colloid can be obtained by starting with silica colloids of a narrow size distribution, which is almost always the case using the methods disclosed by Chen et al, supra.

To grow secondary bio-ceramic layers such as hydroxyapatite onto the nanoparticles, collagen-coated particles were placed in a simulated body fluid containing ions of sodium, potassium, magnesium, calcium, chlorine and acidic groups such as HCO₃^{-, HPO}₄³¹ and S04⁻, at a temperature of 37.5° C. The hydroxyapatite formed is nanocrystalline and can be detected using X-ray diffraction, FTIR and electron microscopy.

Method of presenting Biological Information to a Cell or a Tissue

Further, the invention provides a method of presenting biological information to a cell or a tissue including contacting the three-dimensional construct of the invention with the cell or the tissue to present the biological information and thereby affecting the at least one characteristic of the cell or the tissue.

The three-dimensional construct of the invention can be contacted with the cell or the tissue by any methods known in the art such as, for example injecting or otherwise administering the three-dimensional construct to a body or a cavity in the body, contacting the body with an object molded from cured or gelled three-dimensional construct, and contacting the body with an auxiliary surface which has a coating made from the three-dimensional construct.

The term “coating”, as used herein, includes coatings that completely cover a surface, or portion thereof (e.g., continuous coatings, including those that form films on the surface), as well as coatings that may only partially cover a surface, such as those coatings that after drying leave gaps in coverage on a surface (e.g., discontinuous coatings). The later category of coatings may include, but is not limited to a network of covered and uncovered portions and distributions of the three-dimensional construct or the nanoparticles on a surface which may be porous or have partitions. In some embodiments, the coating preferably forms at least one layer of the three-dimensional construct or at least one layer of the nanoparticles on the surface which has been coated, and is substantially uniform. However, when the coatings described herein are described as being applied to a surface, it is understood that the coatings need not be applied to, or that they cover the entire surface. For instance, the coatings will be considered as being applied to a surface even if they are only applied to modify a portion of the surface.

The term “auxiliary surface” as used herein means a surface coated or otherwise covered with the three-dimensional construct of the invention or with the nanoparticles. Non-limiting examples of the auxiliary surface are a polymer, a carbonaceous material, a wool, a glass, a ceramic, and a metal. The auxiliary surface can be in a shape of a mesh, a fiber, a sheet, a sponge, a layer, a pattern, and a pre-formed object. Examples of polymers suitable for auxiliary surfaces include biodegradable polymers, non-biodegradable water-soluble polymers, non-biodegradable non-water soluble polymers, lipophilic moieties, and biopolymers. In a way of example how auxiliary surface can be utilized in the invention, the nanoparticles comprising silicone oxide functionalized with amine groups and coated with a monomolecular layer of each collagen and hydroxyapatite dispersed in alginate gel can be placed on a mesh or a fiber can be extruded from a polymeric composition including the above described silicone oxide nanoparticle or the three dimensional construct comprising the above described silicone oxide nanoparticle in alginate gel.

In certain embodiments, the diameter, the biological information and the density are selected to affect the at least one characteristic of the cell or the tissue such as, for example, proliferation and differentiation. Non-limiting examples of the three-dimensional construct of the invention are described above. Preferred constructs are silicone oxide functionalized with amine groups and coated with hydroxyapatite, silicone oxide functionalized with amine groups and coated with poly(acryl amine), silicone oxide functionalized with amine groups and coated with a monomolecular layer of each collagen and hydroxyapatite, and silicone oxide functionalized with amine groups and coated with a monomolecular layer of each poly(acryl amine), collagen and hydroxyapatite. Other preferred constructs include any one of the above layer coated onto the nanoparticle comprising at least one of poly(lactic acid), poly(lactic-co-glycolic acid), and poly(anhydride). The three-dimensional construct of the invention can further include a cell as described above.

Additionally, the invention provides a method of administering nanoparticles to a cell to cause a desirable pharmaceutical effect, the method comprising providing nanoparticles, optionally providing an auxiliary surface, wherein the auxiliary surface is a polymer, a carbonaceous material, a wool, a glass, a ceramic, or a metal and wherein the auxiliary surface is in communication with the nanoparticle and contacting the cell with the nanoparticle. Administering nanoparticles can be done by, for example, ways known in the art such as, for example, injecting, swallowing, inhaling and inserting the nanoparticles in a suitable pharmaceutically acceptable media including a solution, a gel or a solid surface as the auxiliary surface. The desirable pharmaceutical effect is to prevent, to diagnose, to improve or to cure a condition.

The invention will be illustrated in more detail with reference to the following Examples, but it should be understood that the present invention is not deemed to be limited thereto.

### EXAMPLES

### Example 1

### Preparation of Functionalized Silica Nanoparticles (FSNP)

Silica particles (SNPs) having 600 nm in diameter were prepared using a modified Stober process. (See W.Stoeber, A. Fink, Controlled Growth of Monodispersed Silica Spheres in the Micron Size Range, J. of Colloid and Interface Science, 26, 62-69, (1968)). 50 ml of a 364 mM tetraethylorthosilicate (TEOS)/ethanol suspension were added to a separate flask containing a 50 ml solution of ammonium hydroxide (11.7 g) and de-ionized water (14.4 g) in ethanol. This 100 ml mixture was stirred for two hours. Following stirring, 75 ml of the resultant 100 ml suspension was saved for amine modification to prepare amine functionalized silica nanoparticles. The remaining 25 ml were washed 3 times with de-ionized water with intermediate centrifugation, and then saved for further experimentation and characterization.

The surface of the 600 nm SNPs was functionalized by reaction of the surface with aminopropyltriethoxysilane (APS) to prepare amine functionalized silica nanoparticles (Amine FSNP). (See K. Suzuki, S. Siddiqui, C. Chappell, J. A. Siddiqui, Modification of Porous Silica Particles with Poly(Acrylic Acid), Polym. Adv. Technol., 11,92-97, (2000)). Initially,75 ml of the 100 ml SNP suspension were adjusted to pH4 using IN acetic acid. Then, Iml of APS was added to the suspension and the suspension was stirred for 30 minutes. Next, the suspension, while being refluxed using a condenser, was heated to 140° C. over 80 minutes before cooling down to 80° C. over 20 minutes. The condenser was then removed, and the solvent was evaporated at 135° C. until 75 ml of suspension remained. The suspension was washed three times with ethanol with intermediate centrifugation. Following washing, 50 ml of the resultant 75 ml Amine FSNP suspension were saved for poly(acrylic) acid functionalization (see Example 2). The remaining 25 ml were saved for further experimentation and characterization.

### Example 2

### Preparation of poly(acrylic) acid functionalized silica nanoparticles (SiO₂—NH₂—PAA or PAA FSNP)

The surface of the Amine FSNP (as described in Example 1) was further functionalized through electrostatic adsorption of poly(acrylic) acid (PAA). (See R. Denoyel, J. C. Glez, P. Trans, Grafting γ-Aminopropyltriethoxysilane onto silica: Consequence of Polyacrylic Acid Adsorption, Colloids and Surfaces A: Physicochemical and Engineering Aspects, 197, (2002), 213-233).

A PAA/ethanol solution was prepared by mixing 1ml of PAA with 10 ml of ethanol, which was then filtered using a 200 m syringe filter. 10 ml of the filtered solution were added to 50 mi of the Amine FSNP/ethanol suspension. Following two hours of stirring, the suspension was washed 3 times with de-ionized water with intermediate centrifugation. 25 ml of the resultant 50 ml PAA FSNP suspension were saved for collagen modification (see Example 4). The remaining 25 ml were saved for further experimentation and characterization.

### Example 3

### Particle Characterization

Scanning Electron Microscopy (SEM) and Energy Dispersive Spectroscopy (EDS) were performed using a JEOL 6300F FEG HRSEM (JEOL Ltd., Tokyo, Japan) equipped with a PGT EDS System (Oxford Instruments, PLC, Oxford, UK). Samples were prepared by placing a drop of particle suspension onto an aluminum stud covered with carbon tape. The solvent was then evaporated in a drying oven at 70° C. The dried stud containing dried particles was coated with Au/Pd using a sputter coater. The samples were analyzed at an accelerating voltage of 51 kV.

Zeta Potential Measurements were performed using a ZetaSizer 3000 HSA analyzer (Malvern Instruments, Southborough, Mass.). Samples were prepared by diluting 4 ml of particle suspension with 40 ml of de-ionized water. The pH of the suspension was adjusted to the desired values before each measurement using ammonium hydroxide and acetic acid.

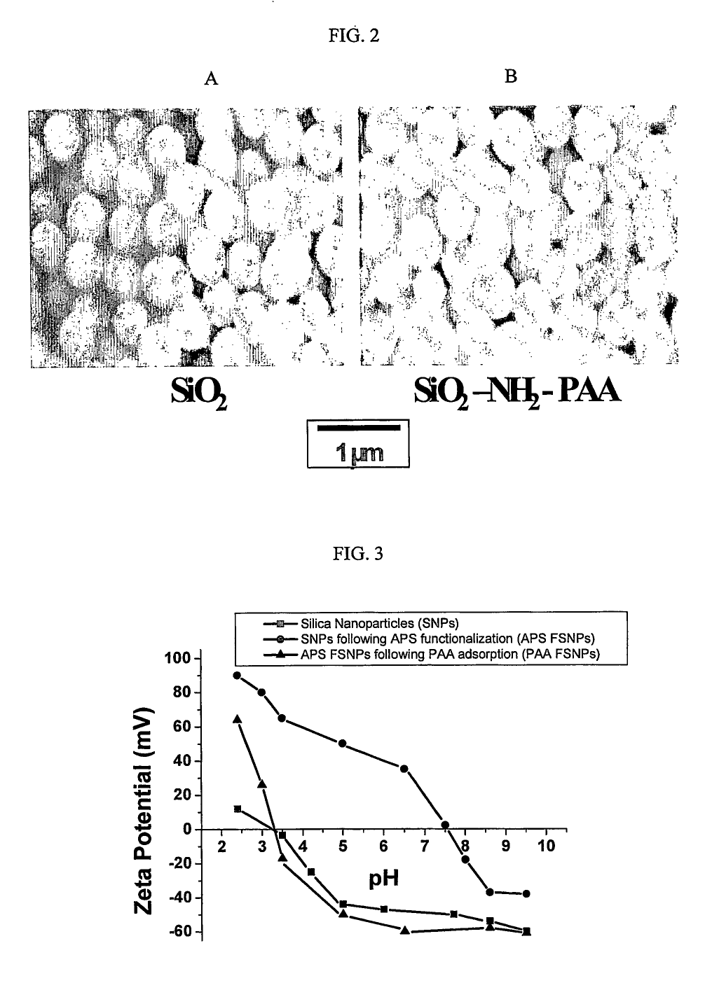

SEM micrographs of the SNPs and PAA FSNP are shown in Fig. 2. The SNP particles are uniform in size with an average diameter of 600 nm. The functionalized PAA FSNP particles had the same size and shape. The molecular coating was too thin to be discerned by SEM.

Zeta potential measurements as a function of pH for the SNPs, Amine FSNP, and PAA FSNP are presented in FIG. 3. The point of zero charge (PZC) is defined at a certain pH value when the zeta potential is zero. For the above three particle types, PZC is at pH 2, 7.5, and 3.5, respectively. At intermediate pH values, the surface charge of the SNPs reverses from a negative value prior to amine functionalization to a positive value following amine functionalization. In contrast, the surface charge of the Amine FSNP reverses from a positive value prior to PAA adsorption to a highly negative value following PAA adsorption. This experiment demonstrates that the surface charge of the nanoparticles changes with functionalizing.

### Example 4

### Electrostatic Adsorption of Collagen onto Amine FSNP

A 200 μg/ml collagen solution was prepared by adding 1 ml of a 2 mg/ml collagen solution to 9 ml of de-ionized water. The pH of the collagen solution was adjusted to pH 6 using ammonium hydroxide and acetic acid.

The pH of the 25 ml of Amine FSNP suspension was adjusted to 6 using ammonium hydroxide and acetic acid. 8.33 ml of the 200 μg/ml collagen solution were then added. Next, the pH of the collagen/Amine FSNP mixture was adjusted to 7.4 to allow electrostatic adsorption of collagen onto the Amine FSNP surface. Following 20 hours of stirring the mixture was washed three times with de-ionized water with intermediate centrifugation.

### Example 5

### Electrostatic adsorption of collagen to PAA FSNP

The pH of the 25 ml of PAA FSNP suspension was adjusted to 6 using ammonium hydroxide and acetic acid. 8.33 ml of the 200 μg/ml collagen solution was then added. Next, the pH of the collagen/PAA FSNP mixture was adjusted to 4.7 to allow electrostatic adsorption of collagen to the PAA FSNP surface. (See N. Barbani, L. Lazzeri, Bioartificial Materials Based on Blends of Collagen and Poly(Acrylic Acid), J. of Applied Polymer Science, v72, 971-976, (1999)). Following 20 hours of stirring the mixture was washed three times with de-ionized water with intermediate centrifugation.

### Example 6

### Particle Characterization

SEM and zeta potential measurements of the FSNP following collagen adsorption was performed using the procedure outlined in Example 3. SEM micrographs of PAA FSNP with electrostatically adsorbed collagen are shown in FIG. 4. Compared to the particles in FIG. 2, same size and shape are found despite the additional collagen adsorption. Therefore, the layer of collagen molecules is too thin to be discerned by SEM.

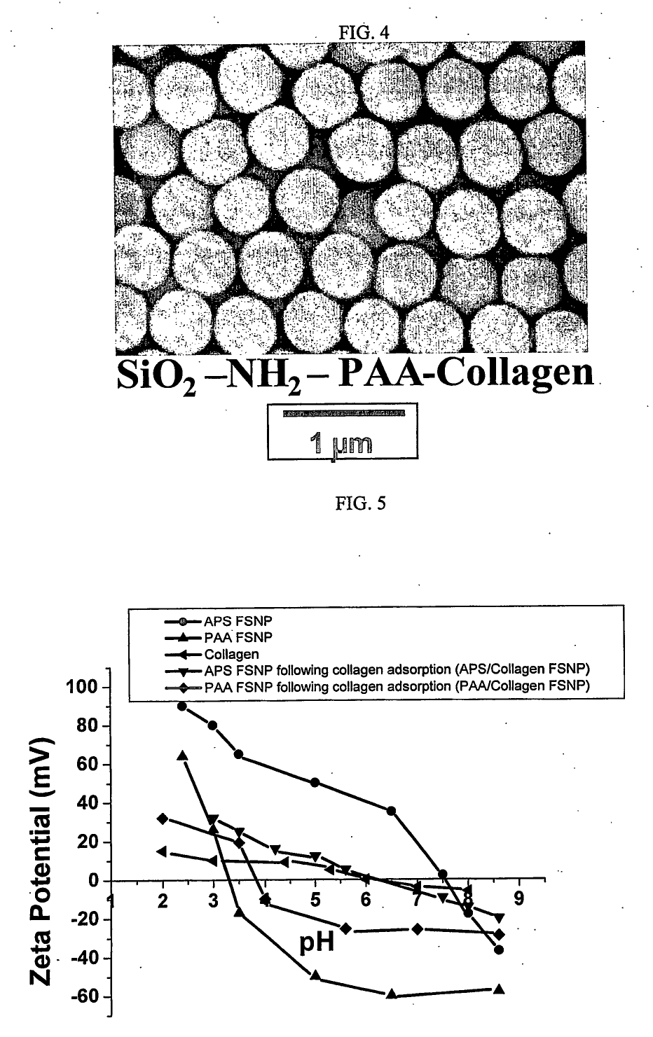

Zeta potential measurements as a function ofpH for collagen, Amine FSNP, PAA FSNP, Amine FSNP following collagen adsorption, and PAA FSNP following collagen adsorption are shown in FIG. 5. The PZC of collagen is at pH 6.2. Following collagen adsorption, the PZC of the Amine FSNP coincides with that of collagen, indicating a complete coverage of the NP by collagen. The PZC of the PAA FSNP after collagen adsorption falls between that of PAA-FSNP and collagen, indicating the effect of collagen adsorption but perhaps an incomplete coverage.

The quantification of collagen surface coverage was performed using the MicroBCA Assay Technique (see Micro BCA Protein Assay Kit, U.S. Pat. No. 4,839,295). In this analysis, the collagen concentration for each sample was determined by comparison with a constructed calibration curve of collagen concentration. The procedure for these measurements is outlined by the manufacturer in the Pierce MicroBCA Assay instruction manual (see K. Suzuki et al., Modification of Porous Silica Particles with Poly(Acrylic Acid), Polym. Adv. Technol., 11,92-97, (2000). Spectroscopy measurements were performed using a Beckman DU 640B Spectrophotometer (Beckman Coulter, Fullerton, Calif.). The collagen surface coverage for each sample was calculated using the measured collagen concentration and the sample surface area were estimated. Following reaction of the suspended particle surface with the BCA working reagent, the suspension was centrifuged. The supernatant, i.e., the activated working reagent, was analyzed by spectroscopy, and the absorption measurement was compared to the constructed calibration curve to determine the concentration of collagen. The settled particles were dried, and the mass was measured using a mass balance. The sample surface area was estimated using the sample mass, the particle size, and the density of amorphous silica (2.2 g/cm³).

Table 1 shows the results of collagen surface quantification using the Micro BCA Assay technique. The amount of collagen surface coverage was 0.09±0.01 μg/cm² for both the Amine FSNP and the PAA FSNP following electrostatic adsorption of collagen.

TABLE 1
Measurements of Collagen Surface
Coverage using the MicroBCA Assay

| FSNP Type | Collagen Surface Coverage |
|---|---|
| Amine FSNP | 0.09 ± 0.01 μg/cm² |
| PAA FSNP | 0.09 ± 0.01 μg/cm² |

### Example 7

### Hydroxyapatite (HAp) coating of FSNP Following Collagen Adsorption

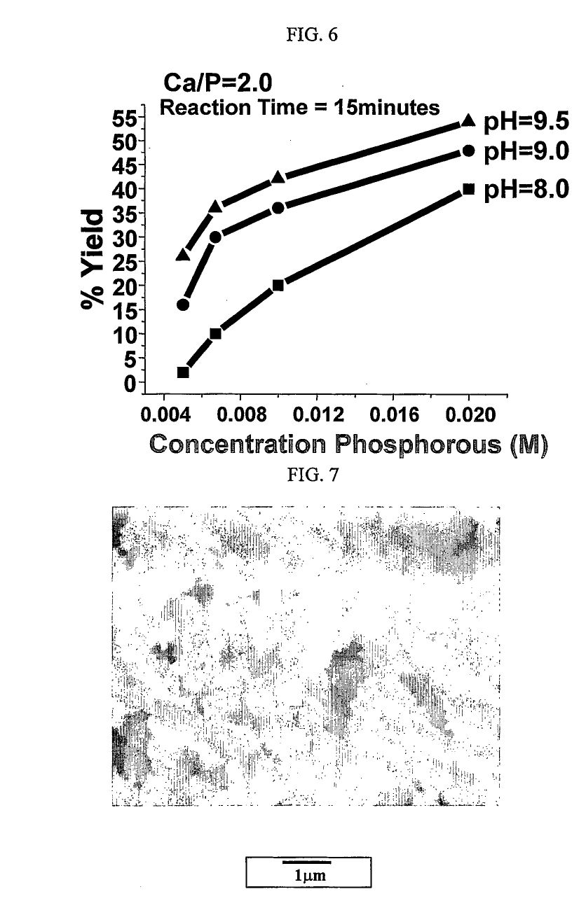

HAp was precipitated from a solution containing calcium-nitrate-tetrahydrate and ammonium phosphate (the reactants) fixed at the 2:1 molar ratio, which is near the Ca:P ratio in HAp (1.67). As shown in FIG. 6, the amount of precipitated HAp, expressed as yield, can be controlled by varying reactant concentrations and pH. The amount increases with increasing both the pH and reactant concentration.

5 ml of a 6 mg/ml PAA/collagen FSNP mixture prepared using the procedure described in Example 5, was added to 400 ml of a 0.014M calcium-nitrate-tetrahydrate /water solution containing 400 mg of HEPES buffer and 3 ml of 1N ammonium hydroxide. The pH of the mixture was then adjusted to 8.0 using ammonium hydroxide and acetic acid. Following pH adjustment, 10 ml of a 0.05 M ammonium phosphate solution was added dropwise over 5 minutes. The solution was then stirred for 10 minutes. Following stirring, the particles were washed 6 times with de-ionized water with intermediate centrifugation.

### EXAMPLE 8

### Particle Characterization

The SEM procedure, the EDS analysis, and zeta potential measurements of the NPs with collagen and HAp coatings were performed using the procedure described in Example 3.

A SEM micrograph of the PAA FSNP following sequential coating with collagen and HAp is shown in FIG. 7. The nodular formation on the particles is due to HAp coating since it is absent in all other figures talken prior to coating with HAp.

The EDS analysis of PAA FSNP following collagen adsorption and HAp coating is shown in FIG. 9. The analysis reveals an elemental composition of calcium and phosphorous characteristic of HAp. The Si peak is due to the presence of SNP particles. The C peak is due to contamination (e.g., a carbon tape and atmosphere). The Na peak is due to contamination (e.g., water and insufficient PAA filtration).

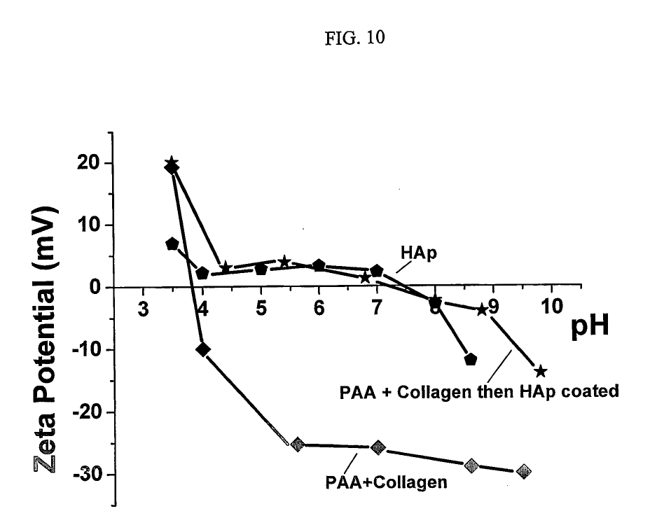

Zeta potential measurements as a function of pH for HAp, PAA FSNP following collagen adsorption, and PAA FSNP following collagen adsorption and HAp coating are shown in FIG. 10. The PZC of HAp is at pH 7.4. Following collagen adsorption, the PZC of PAA FSNP, with collagen adsorption, nearly coincides with this value. This indicates that there is a complete coverage of HAp on the NPs.

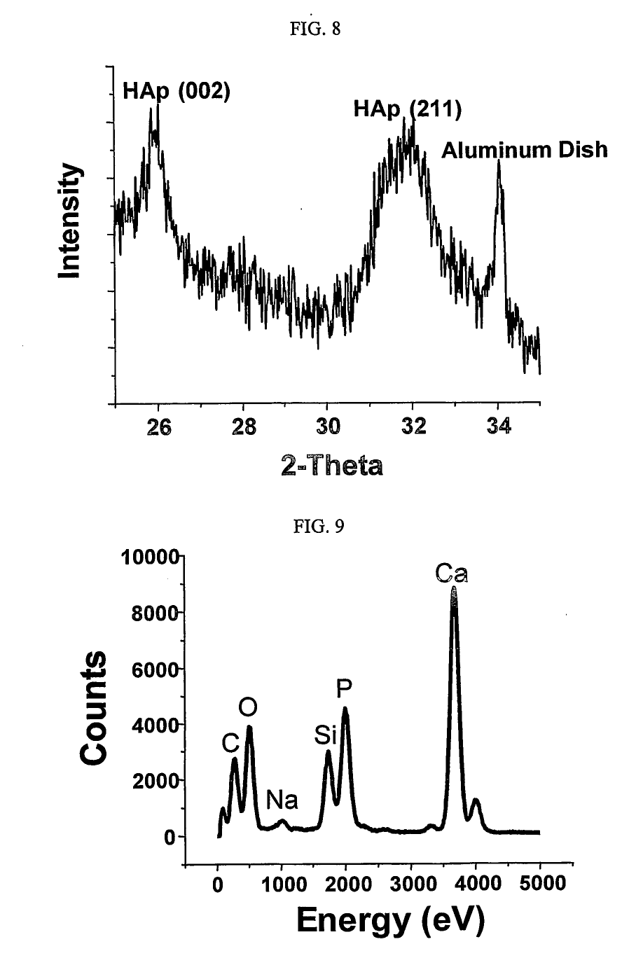

The X-ray Diffraction (XRD) analysis was performed using a Rigaku Miniflex Diffractometer (Rigaku Intercational Corp, Tokyo, Japan). Samples were prepared by drying the particle solution on an aluminum sample holder. The 2-Theta range was from 25 to 35° at a scanning speed of 0.02° /min. An XRD pattern of PAA FSNP following collagen adsorption and HAp coating is shown in FIG. 8. It reveals the (002) and (211) reflections characteristic of HAp. The sharp peak marked as “aluminum dish” is from the sample holder.

### Example 9

### Dispersion of HAp/Collagen Coated FSNP in Alginate Gel

This experiment illustrates how FSNP can be immobilized in a biologically relevant scaffold, such as an alginate gel. The alginate solution and the alginate gel were prepared using guidance from Stevens et al., A Rapid-Curing Alginate Gel System: Utility in Periosteum Derived Cartilage Tissue Engineering, Biomaterials, 25, 887-894, (2004).

A 1 wt % alginate/water solution was prepared by mixing 100 mg alginate with 10 ml water. The mixture was then heated to 90° C. for 10 minutes in order to dissolve the alginate. The solution was then cooled to room temperature.

Next, 10 ml of a 7 mg/ml HAp/collagen coated FSNP mixture were added to 10 ml of the 1 wt % alginate/water solution. The resulting mixture was then stirred for 5 minutes.

The HAp/collagen coated FSNP/Alginate mixture was added to 10 ml of a 0.1 M calcium-nitrate -tetrahydrate/water solution. Gelation occurred immediately.

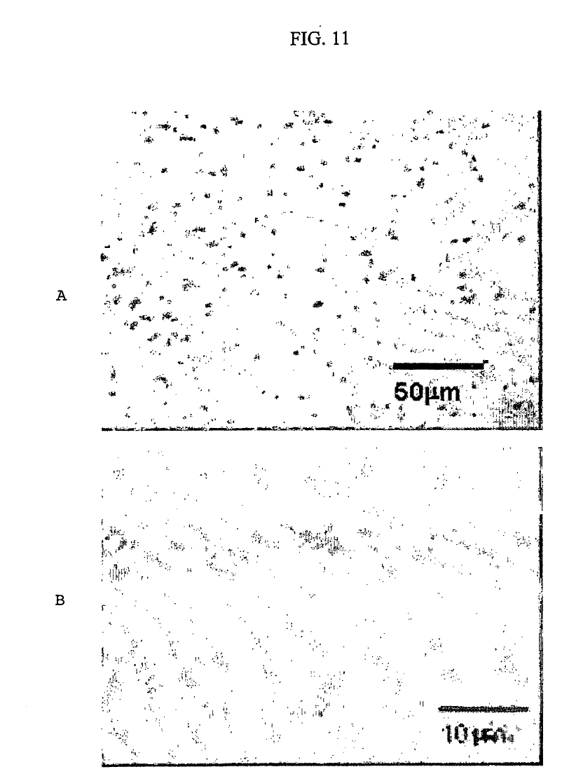

Optical microscopy was performed using a LECO OLYMPUS PMG3 Optical Microscope (Leco Corp, St. Joseph, Mich.). Samples were prepared by smearing the HAp/Collagen coated FSNP/Alginate solution onto a glass slide. The glass slide was then dipped into the 0.1M calcium-nitrate-tetrahydrate solution to cause gelation. Results are presented in FIG. 11. The HAp coated FSNP are dispersed in the gel with minor amounts of agglomeration.

Gelation of HAp/collagen coated FSNP/alginate mixture can be achieved without use of calcium salt. 5 ml of a 3.5 mg/ml HAp/collagen coated PAA-FSNP mixture were added to 10 ml of a 2 wt % alginate/water solution. The pH of the resulting mixture was adjusted using ammonium hydroxide and acetic acid. The effect of pH on the rate of gelation was monitored. Following 24 hours of stirring at pH 7.4, the HAp coated FSNP/alginate mixture remained fluid. When the pH was adjusted to pH 5, the mixture solidified within 2 hours.

### Example 10

### Electrostatic Adsorption of HAp to FSNP

400 ml of a 0.014M calcium-nitrate-tetrahydrate /water solution containing 400 mg of HEPES buffer and 3 ml of IN ammonium hydroxide were adjusted to pH8.0 using ammonium hydroxide and acetic acid. Following pH adjustment, 10 ml of a 0.05M ammonium phosphate solution was added dropwise over 5 minutes. The mixture was then stirred for 10 minutes. Following stirring, HAp was washed 6 times with de-ionized water with intermediate centrifugation (HAp/water suspension). 5 ml of a 2 mg/ml HAp/water suspension were adjusted to pH6. Separately, 4 ml of a 2 mg/ml PAA FSNP/water suspension (see Example 2) were adjusted to pH 6. The HAp/water suspension was then added dropwise to the PAA FSNP/water suspension and the mixture was stirred for 12 hours. Following 8 hours of mixing, HAp crystals were observed by SEM to be adsorbed to the PAA FSNP surface.

While the invention has been described in detail and with reference to specific examples thereof, it will be apparent to one skilled in the art that various changes and modifications can be made therein without departing from the spirit and scope thereof.

What is claimed is:

1. A three-dimensional construct comprising:
a polymeric matrix; and
a nanoparticle comprising a structure and a chemical functional group attached to the structure, wherein the nanoparticle has a diameter of about 5 nm to about 10 microns and is (a) coated with a monomolecular layer comprising biological information and (b) dispersed in the polymeric matrix at a density of at least 0.01 vol %.

2. The three-dimensional construct of claim 1, wherein the monomolecular layer is attached to the nanoparticle by at least one of covalent bonding, hydrogen bonding, ionic bonding, Van der Waals forces and ligand-substrate binding.

3. The three-dimensional construct of claim 1, wherein the monomolecular layer comprises a plurality of sequentially arranged monomolecular layers, wherein an innermost monomolecular layer is attached to the nanoparticle and an outermost monomolecular layer is external to the innermost layer, provided that (a) the innermost monomolecular layer is attached to the nanoparticle by at least one of covalent bonding, hydrogen bonding, ionic bonding, Van der Waals forces and ligand-substrate binding.

4. The three-dimensional construct of claim 1, wherein the plurality of monomolecular layers includes (a) the innermost monomolecular layer, (b) an intermediate monomolecular layer and (c) the outermost monomolecular layer, wherein the intermediate monomolecular layer is attached to at least one of the innermost monomolecular layer and the outermost monomolecular layer, provided that at least two of the plurality of the monomolecular layers are attached to each other by at least one of covalent bonding, hydrogen bonding, ionic bonding, Van der Waals forces and ligand-substrate binding.

5. The three-dimensional construct of claim 1, wherein the biological information is a member selected from the group consisting of a biomolecule, a polymer, and a bone substitute.

6. The three-dimensional construct of claim 5, wherein the biomolecule is a member selected from the group consisting of a bioactive polypeptide, a polynucleotide coding for the bioactive polypeptide, a cell regulatory small molecule, a peptide, a protein, an oligonucleotide, a nucleic acid, a poly(saccharide), an adenoviral vector, a gene transfection vector, a drug, and a drug delivering agent.

7. The three-dimensional construct of claim 6, wherein the bioactive polypeptide is a growth factor and such growth factor is a member selected from the group consisting of an epidermal growth factor, an acidic fibroblast growth factor, a basic fibroblast growth factor, a glial growth factor, a vascular endothelial growth factor, a nerve growth factor, a chondrogenic growth factor, a platelet-derived growth factor, a transforming growth factor beta, an insulin-like growth factor, a hepatocyte growth factor, a brain derived growth factor, bone morphogenic proteins and osteogenic proteins.

8. The three-dimensional construct of claim 5, wherein the polymer is a member selected from the group consisting of poly(carboxylic acid), poly(sulphonic acid), poly(lysine), and poly(allylamine).

9. The three-dimensional construct of claim 8, wherein the poly(carboxylic acid) is poly(acrylic acid).

10. The three-dimensional construct of claim 1, wherein the chemical functional group is a member selected from the group consisting of an amine group, a hydroxyl group, a carboxy group, an —OSO₃H group, a —SO₃H group, a —SH group, an —OCN group, a phosphorous group, an epoxy group, a vinylic moiety, a silane coupling agent, an acrylate, a methylacrylate, a metal alkoxy group, and derivatives thereof.

11. The three-dimensional construct of claim 5, wherein the bone substitute is a member selected from the group consisting of a calcium phosphate, a bioactive glass composition and a bioceramic.

12. The three-dimensional construct of claim 11, wherein the calcium phosphate is a member selected from the group consisting of hydroxyapatite, tricalcium phosphate, tetracalcium phosphate, and octacalcium phosphate.

13. The three-dimensional construct of claim 1, wherein the structure is an inorganic structure or an organic structure.

14. The three-dimensional construct of claim 13, wherein the inorganic structure is a member selected from the group consisting of an oxide, a nitride, a carbide, calcium silicate, calcium phosphate, calcium carbonate, a carbonaceous material, a metal, and a semiconductor.

15. The three-dimensional construct of claim 14, wherein the oxide is a member selected from the group consisting of Al₂O₃, TiO₂, ZrO₂, Y₂O₃, SiO₂, ferric oxide, ferrous oxide, a rare earth metal oxide, a transitional metal oxide, mixtures thereof and alloys thereof

16. The three-dimensional construct of claim 15, wherein the metal is a member selected from the group consisting of aluminum, gold, silver, stainless steel, iron, titanium, cobalt, nickel, and alloys thereof.

17. The three-dimensional construct of claim 13, wherein the organic structure is a member selected from the group consisting of biodegradable polymers, non-biodegradable water-soluble polymers, non-biodegradable non-water soluble polymers, lipophilic moieties, and biopolymers.

18. The three-dimensional construct of claim 17, wherein the organic structure is a member selected from the group consisting of poly(styrene), poly(urethane), poly(lactic acid), poly(glycolic acid), poly(ester), poly(alpha-hydroxy acid), poly(ε-caprolactone), poly(dioxanone), poly(orthoester), poly(ether-ester), poly(lactone), poly(carbonate), poly(phosphazene), poly(phosphonate), poly(ether), poly(anhydride), mixtures thereof and copolymers thereof.

19. The three-dimensional construct of claim 1, wherein the polymeric matrix is a member selected from the group consisting of alginate, hyaluronic acid, poly(ethylene glycol), poly(vinyl alcohol), collagen, a peptide, poly(ethylene oxide)-b-poly(propylene oxide)-b-poly(ethylene oxide), poly(acrylic acid), and poly(isopropyl amide).

20. The three-dimensional construct of claim 1, wherein the three-dimensional construct is in a form of a gel, a cross-linked polymer, a liquid, a foam, a sponge, a mesh, a solid particulate, a fiber, or a layer.

21. The three-dimensional construct of claim 1, further comprising a cell dispersed within the polymeric matrix, wherein the cell is a member selected from the group consisting of chondroblast, chondrocyte, fibroblast, an endothelial cell, osteoblast, osteocyte, an epithelial cell, an epidermal cell, a mesenchymal cell, a hemopoietic cell, an embryoid body, a stem cell, and dorsal root ganglia.

22. The three-dimensional construct of claim 1, wherein the nanoparticle comprises silicon oxide, the chemical functional group comprises an amine group and the monomolecular layer comprises hydroxyapatite.

23. The three-dimensional construct of claim 22, wherein the monomolecular layer comprises at least one of a monomolecular layer of poly(acrylic acid) and a monomolecular layer of collagen as the intermediate monomolecular layer, provided that the monomolecular layer comprising hydroxyapatite is the outermost monomolecular layer.

24. The three-dimensional construct of claim 22, wherein the polymeric matrix is alginate, poly(ethylene glycol), poly(vinyl alcohol), collagen, a peptide, or poly(ethylene oxide) -b-poly(propylene oxide)-b-poly(ethylene oxide).

25. The three-dimensional construct of claim 1, wherein the nanoparticle comprises at least one of poly(lactic acid), poly(lactic-co-glycolic acid), and poly(anhydride) and the monomolecular layer comprises at least one of the monomolecular layer of poly(acrylic acid) and the monomolecular layer of collagen as the innermost monomolecular layer and the monomolecular layer of hydroxyapatite as the outermost monomolecular layer.

26. A method of presenting biological information to a cell or a tissue, the method comprising:
providing the three-dimensional construct of claim 1; and
contacting the three-dimensional construct with the cell or the tissue to present the biological information and thereby affecting the at least one characteristic of the cell or the tissue.

27. The method of claim 26, wherein the diameter, the biological information and the density are selected to affect the at least one characteristic of the cell or the tissue.

28. The method of claim 26, wherein the monomolecular layer is attached to the nanoparticle by at least one of covalent bonding, hydrogen bonding, ionic bonding, Van der Waals forces and ligand-substrate binding and when the plurality of the monomolecular layers is provided, the monomolecular layers are attached to each other by at least one of covalent bonding, hydrogen bonding, ionic bonding, Van der Waals forces and ligand-substrate binding.

29. The method of claim 26, wherein the biological information is a member selected from the group consisting of a biomolecule, a polymer, and a bone substitute.

30. The method of claim 26, wherein the nanoparticle comprises an inorganic structure or an organic structure.

31. The method of claim 26, wherein the at least one characteristic of the cell or the tissue is proliferation or differentiation.

32. The method of claim 26, wherein the nanoparticle comprises silicon oxide, the chemical functional group comprises an amine group and the monomolecular layer comprises hydroxyapatite.

33. The method of claim 32, wherein the monomolecular layer comprises at least one of a monomolecular layer of poly(acrylic acid) and a monomolecular layer of collagen as the intermediate monomolecular layer, provided that the monomolecular layer comprising hydroxyapatite is the outermost monomolecular layer.

34. The method of claim 33, wherein the polymeric matrix is an alginate, poly(ethylene glycol), poly(vinyl alcohol), collagen, a peptide, or poly(ethylene oxide)-b-poly(propylene oxide) -b-poly(ethylene oxide).

35. The method of claim 33, wherein the hydroxyapatite is deposited onto collagen from an aqueous mixture comprising calcium nitrate tetrahydrate and ammonium phosphate at a molar ratio about 1.5 to about 2 and pH of about 7 to about 9.5.

36. The method of claim 35, wherein the molar ratio is equal 2.

37. The method of claim 26, wherein the nanoparticle comprises at least one of poly(lactic acid), poly(lactic-co-glycolic acid), and poly(anhydride) and the monomolecular layer comprises at least one of the monomolecular layer of poly(acrylic acid) and the monomolecular layer of collagen as the innermost monomolecular layer and the monomolecular layer of hydroxyapatite as the outermost monomolecular layer.

38. The method of claim 27, further comprising contacting the three-dimensional construct of claim 1 with an auxiliary surface prior to contacting the three-dimensional construct with the cell or the tissue.

39. The method of claim 38, wherein the auxiliary surface is a member selected from the group consisting of a polymer, a carbonaceous material, a wool, a glass, a ceramic, and a metal.

40. The method of claim 39, wherein the auxiliary surface is in a shape of a mesh, a fiber, a sheet, a sponge, a layer, a pattern, and a pre-formed object.

41. The method of making the three-dimensional construct of claim 1, the method comprising:
providing the polymeric matrix;
providing an unprocessed nanoparticle;
making the nanoparticle by contacting the unprocessed nanoparticle with a carrier of biological information to form the monomolecular layer; and
dispersing the nanoparticle in the polymeric matrix at the density of at least 0.01 vol. % and thereby making the three-dimensional construct.

42. The method of claim 41, further comprising providing a hardening agent.

43. A nanoparticle comprising:
a structure, said structure is being a member selected from the group consisting of silicon oxide functionalized with a chemical functional group, poly(lactic acid), poly(lactic-co-glycolic acid), and poly(anhydride);
a monomolecular layer of hydroxyapatite; and
optionally a monomolecular layer of poly(acrylic acid) and/or a monomolecular layer of collagen, wherein the structure is coated with the monomolecular layer of hydroxyapatite and optionally with the monomolecular layer of poly(acrylic acid) and/or the monomolecular layer of collagen, provided that the monomolecular layer of hydroxyapatite is an outermost monomolecular layer.

44. The nanoparticle of claim 43, wherein the chemical functional group is a member selected from the group consisting of an amine group, a hydroxyl group, a carboxy group, an —OSO₃H group, a —SO₃H group, a —SH group, an —OCN group, a phosphorous group, an epoxy group, a vinylic moiety, a silane coupling agent, an acrylate, a methylacrylate, a metal alkoxy group, and derivatives thereof.

45. The nanoparticle of claim 43, further comprising an auxiliary surface, said auxiliary surface is a member selected from the group consisting of a polymer, a carbonaceous material, wool, glass, ceramic, and a metal, and wherein said carrier is in communication with the nanoparticle.

46. The nanoparticle of claim 46, wherein the auxiliary surface is in a form of a gel, a liquid, a foam, a solid, a fiber, a mesh, a sheet, a sponge, a pattern, and a pre-formed object.

47. A method of administering the nanoparticle of claim 45 to a cell, the method comprising:
providing the nanoparticle;
optionally providing an auxiliary surface, wherein the auxiliary surface is a member selected from the group consisting of a polymer, a carbonaceous material, a wool, a glass, a ceramic, and a metal and wherein the auxiliary surface is in communication with the nanoparticle; and
contacting the cell with the nanoparticle and thereby administering the nanoparticle.
